(19) United States
(12) Patent Application Publication
(10) Pub. No.: US 2019/0175076 A1
(43) Pub. Date: Jun. 13, 2019

(54) POSTURE IMPROVEMENT DEVICE, SYSTEM AND METHOD

(71) Applicant: SEATBACK ERGO LTD, Nofit (IL)

(72) Inventors: Or LUSTIG, Nofit (IL); Sigal LUSTIG, Nofit (IL); Tamar SHEINSON, Tel Aviv (IL)

(21) Appl. No.: 16/324,564

(22) PCT Filed: Aug. 10, 2017
(86) PCT No.: PCT/IL2017/050887

§ 371 Date: Feb. 10, 2019

Related U.S. Application Data
(60) Provisional application No. 62/373,356, filed on Aug. 11, 2016.

(51) Int. Cl. A61B 5/11; A61B 5/00

(52) U.S. Cl. CPC A61B 5/1121; A61B 5/6891; A61B 5/486; A61B 2562/0219; A61B 5/7455; A61B 5/742; A61B 5/1116

(57) ABSTRACT

A system comprises sensors configured to determine readings relating to a person sitting on a chair; a processor configured to process the readings from the sensors and to estimate an estimated posture of the person based on the sensors' readings; and a haptic feedback device that is located on the chair; wherein said processor is configured to instruct said haptic feedback device to provide haptic feedback to the person based on the estimated posture. A method comprises obtaining readings relating to a person sitting on the chair; estimating an estimated posture of the person based on the readings; selecting a feedback to be provided to the person based on the estimated posture; and issuing a haptic feedback based on the feedback, wherein the haptic feedback is provided to the person by a haptic feedback device that is located on the chair.

### CROSS-REFERENCE TO RELATED APPLICATION

This application claims the benefit of provisional patent application No. 62/373,356 filed Aug. 11, 2016, which is hereby incorporated by reference in its entirety without giving rise to disavowment.

### TECHNICAL FIELD

The present disclosure relates to ergonomics, in general, and to posture improvement, in particular.

### BACKGROUND

The World Health Organization currently ranks physical inactivity as the 4th largest killer globally. Throughout the Globus, the sedentary lifestyle increases in correlation with age.

In the United States of America alone, the average person's sitting time is over 9 hours per day. Back pain is the second most common reason for doctors' visits in the U.S. Over 100 billion dollars are spent every year on back pain in the U.S. (direct medical & associated costs).

It is said that a vast majority of the corporate world spends greater than six hours a day sitting at a desk. During such time, many employees may sit in positions of bad posture, and may even be unaware of the risks they are facing or damage they are causing their bodies.

Most ergonomic products available are static and rely on a user's knowledge of usage or configuration thereof. As an example, an ergonomic chair may be designed to correct one's posture while sitting, but without proper instructions or feedback, the user may become unaware of the incorrectness of her sitting position, which would lead to an ineffective use of the potentially effective product.

### BRIEF SUMMARY

One exemplary embodiment of the disclosed subject matter is a system comprising: a set of sensors configured to determine readings relating to a person sitting on a chair; a processor configured to process the readings from the set of sensors; wherein said processor is configured to estimate an estimated posture of the person based on the readings from the set of sensors; and a haptic feedback device; wherein said haptic feedback device is located on the chair; wherein said processor is configured to instruct said haptic feedback device to provide haptic feedback to the person based on the estimated posture.

Optionally, said haptic device is a vibration motor.

Optionally, said processor is configured to provide to the person a first feedback based on a first estimated posture and a second feedback based on a second estimated posture; wherein the first feedback is a negative feedback inducing a posture modification; and wherein the second feedback is a positive feedback that is provided in response to posture modification from the first estimated posture to the second estimated posture.

Optionally, the system is configured to provide different haptic feedbacks using said haptic feedback device or using an additional feedback device located externally to the chair.

Optionally, said additional feedback device is instructable by said processor via a network; wherein said processor is configured to determine connectivity with the network; wherein said processor is configured to select a feedback device, which is selected from said haptic feedback device and said additional feedback device, based on the determined connectivity; wherein said processor is configured to instruct the selected feedback device to provide a feedback to the person.

Optionally, said additional feedback device is a feedback device of a user device of the person.

Optionally, said haptic feedback device is located on a seat of the chair.

Optionally, said processor is configured to determine a message to be provided to the person, wherein said processor is configured to select a feedback from a plurality of potential alternative feedbacks relaying the message.

Optionally, the potential alternative feedbacks comprise the haptic feedback and at least one of: a written message; a audial feedback; and a visual feedback.

Optionally, system of claim 8, wherein the potential alternative feedbacks comprise one or more alternative content messages, wherein the one or more alternative content messages comprise at least one of: an informative study; an indication of current posture and how to amend the current posture; a visual pressure map; an exercise suggestion; and an immediate stretch suggestion.

Optionally, selection of the feedback from the plurality of potential alternative feedbacks relaying the message is based on outcome history of the person to previous feedbacks.

Optionally, said set of sensors comprise a plurality of pressure sensors and two or more accelerometers; wherein said processor is configured to compute an angle relating to a posture of the person based on readings from said two or more accelerometers, wherein said processor is configured to estimate the estimated posture based on the angle.

Optionally, the system comprises the chair; wherein said set of sensors is embedded within said chair.

Optionally, the system comprising a chair-mountable pad; wherein said chair-mountable pad is selectively mountable on the chair; wherein chair-mountable pad comprising said set of sensors is comprised and said haptic feedback device.

Optionally, said processor is configured to compute one or more physical measurements based on the readings from the set of sensors; wherein said processor is configured to estimate the estimated posture based on the one or more physical measurements; wherein the one or more physical measurements comprise an angle between a lumber of the person and a hip of the person.

Optionally, based on the readings from the set of sensors, said processor is configured to compute a first average angle between a backrest of the chair and a plane parallel to a ground; wherein said processor is configured to compute a second average angle between a seat of the chair and the plane parallel to the ground; wherein said processor is configured to estimate the estimated posture based on the first average angle and the second average angle.

Optionally, said set of sensors comprise a first matrix of sensors located on a seat of the chair, and a second matrix of sensors located on a backrest of the chair.

Another exemplary embodiment of the disclosed subject matter is a method comprising: obtaining, from a set of sensors located on a chair, readings relating to a person sitting on the chair; estimating, by a processor, an estimated posture of the person based on the readings from the set of sensors; selecting a feedback to be provided to the person based on the estimated posture; and issuing a haptic feedback based on the feedback, wherein the haptic feedback is provided to the person by a haptic feedback device that is located on the chair.

Optionally, said estimating is performed by the processor located on the chair.

Optionally, the method comprises determining a current mode selected from an online mode and an offline mode, wherein the online mode is a mode where a remote feedback device is reachable via a network, whereby in the online mode, the remote feedback device can be used to provide the feedback to the person, wherein the offline mode is a mode where the remote feedback device is not reachable via the network; and said selecting is performed based on the current mode.

Optionally, said estimating and said selecting are performed based on rules; said method further comprises in response to changing from the offline mode to the online mode, transmitting posture information to a server and receiving from the server updated rules, whereby affecting future posture estimations or future feedback selections.

Optionally, said selecting is performed based on one or more rules; wherein said method further comprises: monitoring response of the person to the haptic feedback; and updating the one or more rules, whereby personalizing future feedback selections to select effective feedback for the person.

Optionally, said obtaining comprises obtaining readings from at least three accelerometers; wherein said method further comprises computing two angles based on the readings from the three accelerometers; and wherein said estimating is based on the two angles.

Optionally, the two angles comprise: a first average angle between a backrest of the chair and a plane parallel to a ground or the seat of the chair; and a second average angle between a seat of the chair and the plane parallel to the ground.

### THE BRIEF DESCRIPTION OF THE SEVERAL VIEWS OF THE DRAWINGS

The present disclosed subject matter will be understood and appreciated more fully from the following detailed description taken in conjunction with the drawings in which corresponding or like numerals or characters indicate corresponding or like components. Unless indicated otherwise, the drawings provide exemplary embodiments or aspects of the disclosure and do not limit the scope of the disclosure. In the drawings:

FIG. 1A shows an illustration of a chair, in accordance with some exemplary embodiments of the disclosed subject matter;

FIG. 1B shows an illustration of a chair-mountable pad mounted on a chair, in accordance with some exemplary embodiments of the disclosed subject matter;

FIG. 2 shows a schematic illustration showing angle computations using sensors, in accordance with some exemplary embodiments of the disclosed subject matter;

FIG. 3 shows a flowchart diagram of a method, in accordance with some exemplary embodiments of the disclosed subject matter;

FIG. 4 shows a flowchart diagram of a method, in accordance with some exemplary embodiments of the disclosed subject matter;

FIG. 5 shows a flowchart diagram of a method, in accordance with some exemplary embodiments of the disclosed subject matter;

FIG. 6 shows a block diagram of a system, in accordance with some exemplary embodiments of the disclosed subject matter;

FIGS. 7A-7F show illustrations of user interface of an application program, in accordance with some exemplary embodiments of the disclosed subject matter.

### DETAILED DESCRIPTION

One technical problem dealt with by the disclosed subject matter is to improve a posture of a person sitting on a chair.

Another technical problem dealt with by the disclosed subject matter is to detect current posture in an accurate manner, and provide effective feedback to the person.

Yet another technical problem is to allow posture detection and effective feedback using relatively low power consumption.

One technical solution provided by the disclosed subject matter is to utilize a set of sensors located on a chair, either integrated therein or mounted thereon, to detect the posture of the person sitting on the chair. The sensors may include sensors useful for determining pressure exerted on the chair at different locations. In particular, a plurality of pressure points may be monitored in a seat of the chair and a plurality of pressure point may be monitored in a backrest of the chair. In some exemplary embodiments, a matrix of 3×3 or more pressure points may be used for the seat, the backrest, or for both. In some exemplary embodiments, one matrix may be located on the backrest and a second matrix may be located on the seat.

Additionally or alternatively, the sensors may include orientation sensors for detecting orientation of several points on the chair. The orientation sensors may be useful for computing one or more angles relevant for the posture of the person sitting on the chair. For example, the angles may comprise an average angle between the seat of the chair and a plane parallel to the ground, an average angle between the backrest of the chair and a plane parallel to the ground, or the like. As another example, the computed angle may be the angle between a lumber and a hip of the person sitting on the chair. In some exemplary embodiments, the orientation sensors may be accelerometers, which may indicate orientation when the chair is still, based on gravitational forces. In some exemplary embodiments, the accelerometer may be used when the chair is in motion, such as in case the chair is a chair within a car, by deducting external forces, which may be obtained from additional sensors.

In some exemplary embodiments, feedback is provided to the person using a haptic feedback device, such as a vibrating motor, a kinesthetic feedback device, a tactile feedback device, or the like. In some exemplary embodiments, several different forms of haptic feedbacks may be used to relay a different message. For example, a same vibration motor may vibrate intermittently for 3 seconds to relay one message, and vibrate continuously for 3 seconds to relay another message. In addition, there may be several vibration motors used to provide different feedbacks. For example, there may be two motors located on the seat of the chair, one located in the center of the left half of the chair and the other in the center of the right half of the chair. A vibration by the left motor may indicate the person needs to mind his posture relating to his left hand-side (e.g., tilting to the left excessively); a vibration by the right motor may similarly relate to the person's right side; and a simulations vibration by both motors may indicate the person has correctly corrected his posture. As yet another example, a relatively long vibration by both motors may prompt the user to stand up after a determination of a length sit session is determined. As yet another example, a vibration by both motors in a non-continuous manner may indicate bad posture which needs to be corrected.

In some exemplary embodiments, the feedback provided to the person may be negative feedback. The negative feedback may indicate that the person is currently not sitting in a good posture. The negative feedback may indicate to the user that a posture modification is to be made. In some exemplary embodiments, after the person modifies his posture, a positive feedback may be provided. The positive feedback may indicate that the person has correctly modified his posture. In some cases, the positive feedback may be provided even if the person has not completely correct his person, but rather made a modification of a sufficient magnitude (e.g., above a configurable threshold).

In some exemplary embodiments, feedback may be halted for a predetermined timeframe. For example, after three haptic feedbacks provided to the person, no additional haptic feedbacks may be provided within half an hour. Additionally or alternatively, the halting period of the feedback may be generally for the person (regardless of the conduit of and of the message relayed by the feedback), specific for a type of feedback (e.g., no more vibration feedbacks), specific for a conduit of the feedback (e.g., no more feedbacks using the same feedback device), specific for a message relayed by the feedback (e.g., no more feedbacks relating to the current posture, regardless of the conduit), combination thereof, or the like.

Another technical solution provided by the disclosed subject matter is to operate in both an online mode and an offline mode. Offline mode is a mode where network connectivity is limited. For example, when a Bluetooth or other wireless connectivity is disabled. During offline mode, power consumption is reduced in comparison to the online mode, where the network connectivity exists.

In some exemplary embodiments, the apparatus may be configured to make its own determination without requiring online computation or computation on a cloud. In offline mode, the device may perform posture estimation locally on-device and may select a feedback from the local feedback devices, such as integrated vibration motors embedded with the device itself. In online mode, the selection may be performed from additional feedback devices as well, such as sending an email, sending push notification using an application program in a mobile device, sending a text message to a mobile device, sending a message to a wearable device, such as smart glasses, smart watch, or the like. For example, a tracker bracelet may be worn by the person, and may be used to provide written notifications, haptic feedback, or the like, during online mode.

In some exemplary embodiments, when the mode changes from offline mode to online mode, data may be transferred in a batch mode. In some exemplary embodiments, the device may transmit all accumulated data (e.g., sensor data, posture estimation data, feedback provided, or the like). Additionally or alternatively, the device may receive updated data, such as updated rules, configurations or the like.

Yet another technical solution provided by the disclosed subject matter is to monitor responsiveness of the person to the feedback and update the feedback selection rules accordingly. In some cases, feedback history may include feedback provided to the person and responsiveness score of the user to each such feedback. Based on the feedback history, feedback selection may be optimized. In some cases, a message to be relayed to the person may be determined and a feedback may be selected from potential alternative feedbacks relaying the same message. For example, the same message indicating “leaning to the left”, may be provided using haptic feedback, by a written message, using audial feedback, and using visual feedback. In some cases, different content messages may be provided such as textual message indicating of the incorrect posture, the person may be informed of an informative study attesting to the dangerous and adverse affects of such incorrect posture, the person may receive an exercise suggestion to begin performing to strengthen muscles that are relevant to the incorrect posture, the person may receiving an immediate stretch suggestion that may be suitable to improve posture, potentially specifically with relation to the incorrect posture (e.g., stretching the right side of the body to cause the back to be positioned in a more symmetric manner), the person may be shown a visual indication of his current posture and an indication of how to improve the posture, such as by visually emphasizing a body part to be moved, the person may be provided with a visual pressure map showing the pressure sensed by the pressure sensors, or the like. In some cases, some of the messages may be written messages, have embedded videos or links thereto, or the like. In some exemplary embodiments, the outcome of the feedback may be monitored to determine if it had resulted in the desired effect. The desired outcome may be an immediate outcome (e.g., changing the posture) or long-term outcome (e.g., improving muscle strength, and thereby improving posture in the long-run). Based on the outcome, the disclosed subject matter may improve its future feedbacks and provide more efficient feedbacks for the same person. In some exemplary embodiments, the feedback history may be fed into a classifier for training the classifier to predict an outcome score to a feedback. The classifier may be utilized when a new feedback is selected between alternative potential feedbacks to select the feedback with the highest predicted score.

In some exemplary embodiments, based on the estimated posture, a physical change of the chair may be performed to improve posture or reduce risks from the current posture. Hence, instead of alerting the person, the chair may autonomously modify itself to fit the person. As an example, the height of the chair may be modified, so as to improve posture if the person's legs do not reach the ground. As another example, the chair may include an inflatable lumber support pillow, which may be automatically inflated or deflated to suit the estimated posture.

One technical effect of the disclosed subject matter may include the ability to provide feedback to a user, in different modes—both when the device has and does not have connectivity. Estimation is still performed even in the absence of connectivity to a cloud computing platform.

Another technical effect may include the reduced power consumption that is achieved by the disclosed subject matter. By allowing offline mode to provide feedback to the person, such mode may operate continuously with a reduced power consumption as no power is required for wireless connectivity. Such reduced power consumption may enable an apparatus in accordance with the disclosed subject matter to operate for longer periods of time without recharging its battery, when the device is disconnected from an external power source such as a power outlet.

Yet another technical effect may include an improved posture estimation in comparison to other automated methods. In some cases, using a plurality of matrices may provide for a better posture estimation based on relatively high resolution of pressure points on both the seat and the backrest. Additionally or alternatively, sensors used to determine average angles may be useful for providing an accurate posture estimation, taking into account not only pressure exerted by the person when sitting, but also angles of his body.

The disclosed subject matter may provide for one or more technical improvements over any pre-existing technique and any technique that has previously become routine or conventional in the art.

Additional technical problem, solution and effects may be apparent to a person of ordinary skill in the art in view of the present disclosure.

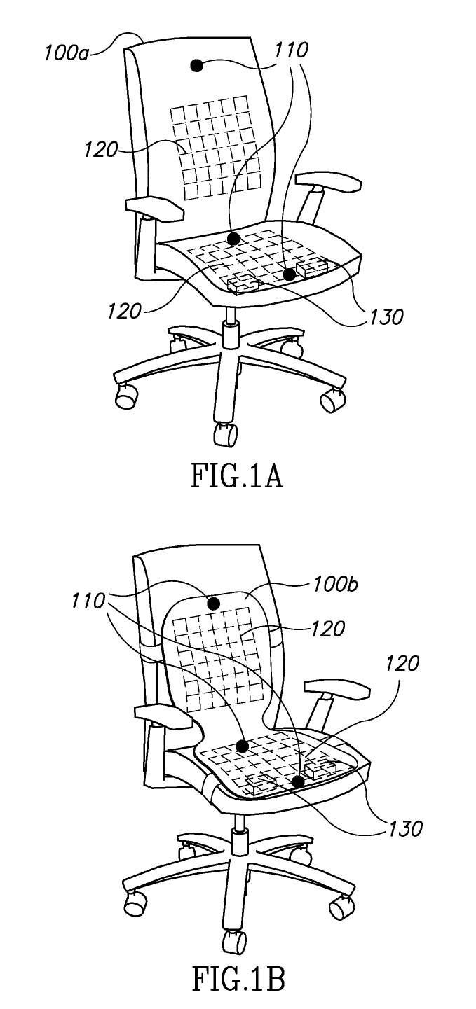

Referring now to FIG. 1 showing an illustration of a chair, in accordance with some exemplary embodiments of the disclosed subject matter.

Chair 100a has embedded therein a set of sensors. The set of sensors comprise Angle-related Sensors 110, a Pressure Sensor Matrix 120 in the seat of Chair 100a and a Pressure Sensor Matrix 120 in the backrest of Chair 100a.

In some exemplary embodiments, Angle-related Sensors 110 may be accelerometers. The accelerometers may be located, for example, at proximity of a top of backrest, at a bottom of backrest or inner border of seat, at an external border of seat, or the like. In some exemplary embodiments, the accelerometers may be located approximately where a symmetry axis of a person sitting on the chair is expected to be located, along a halving axis (not shown) of the backrest and/or along a halving axis (not shown) of the seat.

In some exemplary embodiments, Pressure Sensor Matrix 120 may comprise an N×M matrix of pressure sensors enabling sensing pressure excreted by a person sitting on seat and leaning back against backrest. The matrix may comprise 3×3 sensors, or more, such as 4×4, 6×6, 8×6, 10×8, or the like. In some exemplary embodiments, Pressure Sensor Matrix 120 may comprise N×M distinct sensors. Additionally or alternatively, Pressure Sensor Matrix 120 may comprise a single integrated circuit configured to obtain sensor readings from N×M connected sensors.

Chair 100a may comprise Haptic Feedback Devices 130. A Haptic Feedback Device 130, such as a vibration motor, may be configured to provide a haptic feedback to a person sitting on Chair 100a. In some exemplary embodiments, as a person sitting on Chair 100a may not necessarily lean against backrest, at least a portion of Haptic Feedback Devices 130 may be located on the seat of Chair 100a. Each Haptic Feedback Device 130 may be configured to provide a plurality of different haptic feedbacks, such as vibrations at different frequencies, continuous and non-continuous vibrations, or the like. As an example, Haptic Feedback Device 130 may be configured to vibrate for 1.5 seconds, seize vibrations for 0.5 seconds, and then vibrate again for 1.5 seconds, to relay one message. Different pattern of vibrations and pauses may be used to relay other messages. In some exemplary embodiments, Haptic Feedback Devices 130 may work in synchronization to relay a desired message, such as by vibrating at the same time, by vibrating at disjoint times, or the like.

In some exemplary embodiments, Chair 100a may comprise an inflatable lumber support pillow (not shown). Chair 100a may cause automated inflation or deflation of the inflatable lumber support pillow, such as using a compressor (not shown). In some exemplary embodiments, the automated adjustment of the chair may include setting the height of the chair (e.g., in case of a chair having a telescopic leg) by either lowering or raising the seat of the chair. Additionally or alternatively, the automated adjustment of the chair may include setting an angle of the backseat, such as when such backset has adjustable angle.

In some exemplary embodiments, the automated adjustment may be performed based on physical measurements of the person, such as provided explicitly by the user, or gathered from the sensors and analysis thereof. In some exemplary embodiments, height of the person may be inferred from the pressure sensors from the backseat. For example, the first pressure sensors in the backrest that sense pressure may indicate a length of the lumber of the person. Additionally or alternatively, the weight of the person may be estimated based on the amount of pressure overtime. In some exemplary embodiments, the weight of the person may be based on calculated of average pressure overtime, combined with the angles of the seat and backrest. Additionally or alternatively, estimation as to the amount of pressure exerted by the legs on the surface that is not sensed by the sensors may be computed and utilized. It will be noted that the measured pressure may change over time not only based on changes in weight, but also on changes in weight distribution. If the person puts his leg on the ground, not all of his weight is distributed on the pressure sensors. Monitoring the readings from the pressure sensors over time may be useful in estimating actual weight based on the different pressure measurements and distributions thereof. In some exemplary embodiments, training datasets may be provided based on users' that explicitly provided their physical measurements, and the information may be used to train a supervised machine learning classifier to estimate a weight of another person based solely on the sensor readings.

In some exemplary embodiments, the automated adjustment may be performed based on the physical measurements of the person and in view of known information about the chair. In some cases, expert knowledge about different chairs may be digitized and applied to determine the correct configuration for a chair for a person having specific physical measurements. The automated adjustment may comprise obtaining, using the digitized expert knowledge, the correct configuration, and adjusting the chair from a current configuration to the correct configuration.

It will be appreciated that Chair 100a is exemplified using an office chair. However, the disclosed subject matter is not limited to such an embodiment and any kind of chair may be used. For example, Chair 100a may be a car chair, which may be permanently connected to a power source and not rely on intermittent connection to power source (e.g., for periodically recharging an internal battery).

Referring now to FIG. 1B showing an illustration of a chair-mountable pad mounted on a chair, in accordance with some exemplary embodiments of the disclosed subject matter.

Chair-mountable Pad 100b is mounted on a chair, located on the chair's backrest and seat. Chair-mountable Pad 100b may comprise the same elements described with respect to Chair 100a, including, for example, Angle-related Sensors 110, two Pressure Sensor Matrices 120, and Haptic Feedback Devices 130.

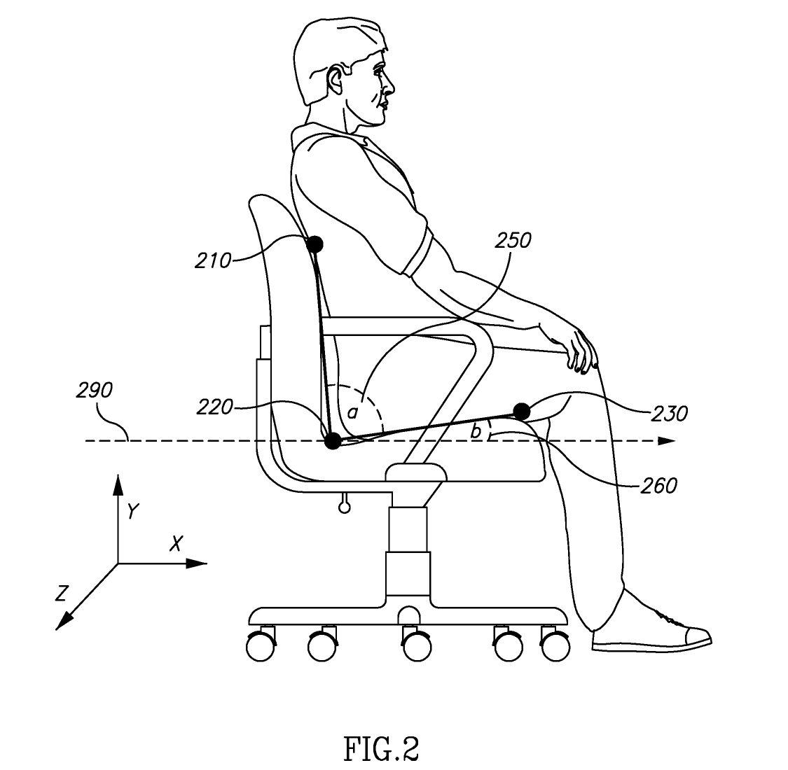

Referring now to FIG. 2 showing a schematic illustration showing angle computations using sensors, in accordance with some exemplary embodiments of the disclosed subject matter.

Accelerometers 210, 220 and 230 may be configured to determine a stationary orientation of backrest and seat. The stationary orientation may be determined based on the division of the gravitational forces measured by the accelerometers at different axes. For example, Accelerometer 210 may be configured to provide (X₁,Y₁,Z₁) forces of a relative top point of backrest, Accelerometer 220 may be configured to provide (X₂,Y₂,Z₂) forces of a relative bottom point of backrest and/or internal point of seat, and Accelerometer 230 may be configured to provide (X₃,Y₃,Z₃) forces of a distal point of seat. Consider the following readings: (0,0.5,0.5) by Accelerometer 210, (0,0,1) by Accelerometer 220, and (0,0.35,0.65) by Accelerometer 230. Such readings may be translated into computing α angle 250 to be 135° and β angle 260 to be 30°. The computation may be performed using the following formula:

$$angle [ deg ] = {tan}^{- 1} ( \frac{{Z}_{i}}{{Y}_{i}} ) - {tan}^{- 1} ( \frac{{Z}_{i + 1}}{{Y}_{i + 1}} ) .$$

In some exemplary embodiments, β angle 260 may be the angle between the seat of the chair and a plane parallel to the ground (290). α angle 250 may be the angle between the backrest of the chair and the seat of the chair. α angle 250 may be computed by first computing an angle between the backrest of the chair and the plane parallel to the ground (290) and subtracting therefrom β angle 260. In the above example, the angle of 165° may be determined and 30° may be subtracted therefrom to calculate α angle 250. In some exemplary embodiments, α angle 250 may be useful in indicating an angle between a lumber and a hip of the person sitting on the chair.

In some exemplary embodiments, the angles being computed are average angles of the surface of the seat and/or backrest, as opposed to an angle in a specific point which may be different due to a skew caused by pressure exerted specifically on that point.

In some exemplary embodiments, other angles that are useful in estimating correctness of posture, may be computed.

It will be noted that the disclosed subject matter is not limited to the use of three accelerometers and such an embodiment is shown as an example only. In some exemplary embodiments, a pair of accelerometers may be used to compute a single angle. Additionally or alternatively, several pairs of accelerometers may be used to compute multiple angles, which may or may not be associated to one another. In some exemplary embodiments, such as in the embodiment of FIG. 2, some pairs of accelerometers may share a common accelerator.

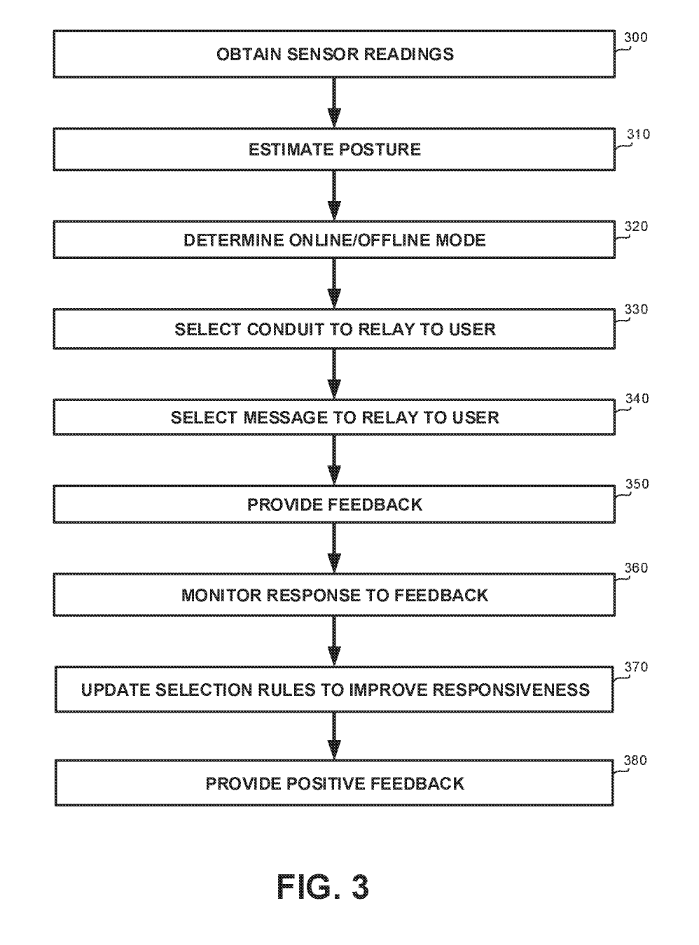

Referring now to FIG. 3 showing a flowchart diagram of a method, in accordance with some exemplary embodiments of the disclosed subject matter. The method may be performed in whole or in part by a processor comprised by an apparatus such as Chair 100a or Chair-mountable Pad 100b.

On Step 300, sensor readings may be obtained. The sensor readings may be obtained from sensors located within the apparatus, such as not requiring wireless or network connectivity to be obtained. The sensors may comprise sensors useful for determining angles, pressure sensors, or the like.

On Step 310, posture of the person may be estimated based on the sensor input. The estimation may be performed by the processor using a supervised classifier, such as a k-means, Support Vector Machines (SVM), or the like, which may be trained to deduce a posture based on input based on training data provided thereto. The training data may comprise sensor readings and correct labels thereof, indicating the posture. The posture estimation may be performed on the apparatus itself without requiring computation by an external computation platform, such as a cloud-based server. In some exemplary embodiments, the classifier may be trained offline and the trained model may be provided to the apparatus to be used locally. In some exemplary embodiments, the potential posture for estimation may include, for example, forward sloping, slump, side reliance, cross legged, no legs support, correct posture with back support, correct posture without back support, standing, or the like. Each posture may be associated with a different severity measurement, a different alleviating stretches or exercises, or the like.

On Step 320, online/offline mode may be determined. In some exemplary embodiments, the mode may be determined by the processor, based on the current situation, such as whether or not there is connectivity. Additionally or alternatively, the processor may force a mode change, such as may force switching from offline mode to online mode to enable data transfer. In some cases, online mode may be reached periodically and after a predetermined period of time, online mode may be forced. Additionally or alternatively, offline mode may be forced to preserve power levels. In case a battery power source is depleting, offline mode may be forced to reduce power consumption and enable longer period of time without charging. In some exemplary embodiments, as long as no feedback is to be provided, offline mode may be preserved. Online mode may potentially be enabled when feedback is desired to be provided using an external feedback device (e.g., mobile device of the person sitting on the chair), or when batch mode data transfer is desired (e.g., reaching predetermined data size for data to be transferred off-device).

On Step 330, a conduit to relay a message to the user may be selected. The conduit may be selected out of the available conduits, such as feedback devices which can be instructed under the current mode. In some cases, some devices may connectable directly via wireless connection such as using Bluetooth protocol. In such a case, some of the potential feedback devices may be available at each point. As an example, consider the device not being connected to the Internet. In such a case, sending an email or transmitting a text message would be conduits that are unavailable. At such time, the device may connected via Bluetooth to an augmented reality glasses, which could be used to provide feedback. In addition, another auxiliary device, such as a smart watch may not be connected and may thus not be available.

On Step 340, a message to be provided to the user may be selected. The message may be selected from a potential alternative messages that are suitable for the estimated posture. In some cases, alternative messages may be informative papers, written explanation about exercises, videos of exercises, textual or visual description of immediate stretch operations, or the like. The message may be a set of vibrations, or other haptic feedbacks that could be understood by the person, based on a predetermined language. Other audial, visual or textual ques may be selected to relay a desired message to the person. In some exemplary embodiments, the selected message may be selected based on the selected conduit of Step 340.

In some exemplary embodiments, the message may be selected based on the physical measurements of the person. For example, the weight and height of the person may be used in a selection of a message. As an example, different stretch exercises may be suggested to people of different heights and weights. In some exemplary embodiments, the physical measurements may indicate a potential health issue caused by the posture, such as predicting which spinal disc is in risk of being involved in an injury. The message may be selected in accordance with such estimation.

In some exemplary embodiments, Steps 330 and 340 may be performed at the same time. Additionally or alternatively, the steps may be performed in reverse order—first selecting a message then selecting conduits that can relay such message.

In some exemplary embodiments, Steps 330 and 340 may be performed based on selection rules. The selection rules may be manually set. Additionally or alternatively, the selection rules may be modified automatically (see Step 370 below) to improve the outcome of sending a feedback. In some cases, the selection rules may also indicate when not to provide any feedback, such as in case of a fourth attempt to send a message for the same posture within a timeframe after the previous three attempts were disregarded. In some cases, the selection rules may be a trained classifier that is trained to predict a score for each potential pair of (message, conduit). The predicted score may be indicative of an estimated effectiveness of using such feedback. The classifier may be re-trained using new observation as for the user's behavior to improve effectiveness for the same person based on feedback history.

On Step 350, the feedback may be provided to the person. The feedback may be provided via the conduit selected on Step 330 and using the message selected on Step 340.

On Step 360, the response to the feedback may be monitored. The response may be monitored in the short-run, such as determining whether the person stands up, changes his posture, or the like. Additionally or alternatively, the response may be monitored in the long-run, such as determining an improvement trend which can be attributed to the user performing exercises that were sent to him.

On Step 370, selection rules may be updated to improve responsiveness. The selection rules may be updated to increase likelihood that the same selection would be performed again if the selection stimulated a positive response, or decrease the likelihood of the same selection, if the person ignored the feedback. Additionally or alternatively, the selection rules may be updated by adding a labeled feature comprising the pair of (message, conduit) used and the label which comprises a score based on the monitored response. The labeled feature may be provided to the classifier for updating the statistical model thereof to improve score prediction for future feedback selections.

On Step 380, in case of a positive response, a positive feedback may be provided to the person. The positive feedback may be provided to indicate to the person that he had corrected the posture, and is currently sitting at a better posture than before.

It will be noted that in some exemplary embodiments, some or all of the determinations may be performed locally on the device or remotely on a remote server, such as on a cloud-based server. As an example, one embodiment may require continuous network access allowing data to be transmitted in real-time to the server and estimation and feedback selection may be performed thereon. In case the feedback is performed using a feedback device within the apparatus, the feedback instruction may be transmitted to the apparatus. Additionally or alternatively, feedback that is provided using external feedback devices may be provided by instructing such devices from the server and without necessarily communicating through the apparatus, to provide the feedback.

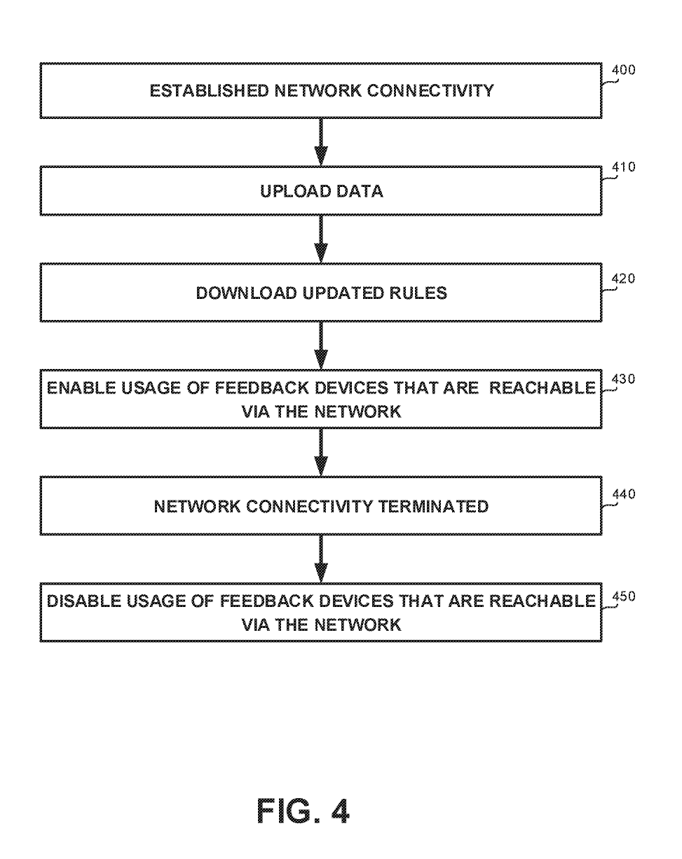

Referring now to FIG. 4 showing a flowchart diagram of a method, in accordance with some exemplary embodiments of the subject matter.

On Step 400, network connectivity is established. Network connectivity may provide a connection between the apparatus on the chair and between a remote server, such as a cloud-based server. Additionally or alternatively, the connectivity may enable communication between the apparatus and the person's mobile device, which may or may not be connected to the remote server. In some cases, the mobile device may serve as an intermediate proxy for providing communication between the server and the apparatus.

On Step 410, data may be uploaded to the server. The data may be uploaded directly or via the mobile device. In some cases, the data may be uploaded to the mobile device and when the mobile device gains access to the network, the data may be transmitted to the server. The data being uploaded may comprise posture history, sensor data, feedback outcome, data relating to monitoring feedback outcome, or the like.

On Step 420, updated rules may be downloaded to the apparatus. The updated rules may comprise updated estimation rules, updated feedback selection rules, or the like. In some cases, the server may train classifiers in an offline manner based on uploaded data and based on initial training data. The server may send the trained classifiers, the statistical model thereof, or the like, to the apparatus to be applied locally without requiring real-time access to the server.

On Step 430, feedback devices that are reachable via the network may deemed available for usage. The feedback devices may not have been usable before network connectivity was established (Step 400). In case a feedback is to be provided, the feedback devices may be used in addition to or instead of the feedback devices that are integrated in the device.

On Step 440, the network connectivity may be terminated. The network connection may be intentionally terminated, such as to preserve power resources, or unintentionally, such as due to external reasons.

On Step 450, the feedback devices that are reachable via the network may no longer be reachable. As a result, the usage of such feedback devices may be disabled and feedback selection may be performed without using such feedback devices.

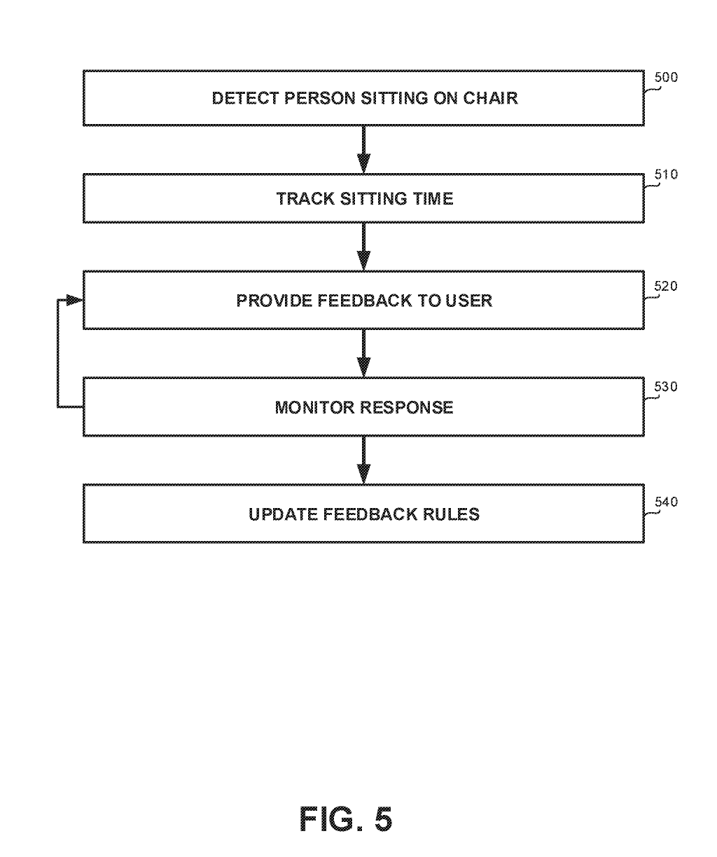

Referring now to FIG. 5 showing a flowchart diagram of a method, in accordance with some exemplary embodiments of the subject matter.

On Step 500, a person sitting on the chair may be detected. The detection may be based on pressure sensors indicating the person is sitting on portions of the chair.

On Step 510, time of the sitting session may be tracked. A cumulative time in which the person is sitting continuously may be tracked.

On Step 520, once a threshold is exceeded, feedback may be provided to the person to indicate he should stand up.

On Step 530, the response of the person may be monitored. Based on the monitored response, additional feedback may be provided (520), such as feedbacks using different conduits, using different content, or the like, in an attempt to stimulate the person to stand up.

On Step 540, feedback rules may be updated. Feedback rules may be updated to reflect which feedbacks promoted response from the person. In some exemplary embodiments, after reaching a predetermined amount of trials, such as three attempts, no additional feedback may be provided for a predetermined time window, such as about 15 minutes, about 30 minutes, or the like. The feedback rules may be updated to set a timer until additional feedback may be provided. In some exemplary embodiments, during the feedback halting time window, no feedback at all may be provided. Additionally or alternatively, during the feedback halting time window, only feedback on the specific posture issue may be halted and feedbacks on other posture issues may still be issued. For example, feedback on an improper posture may be issued, while no additional feedback may be provided regarding the lengthy sitting session.

It will be noted that the above can be applied to feedbacks to other types of sitting-related issues, and particularly to improper posture, and that the above is not limited to lengthy sitting sessions.

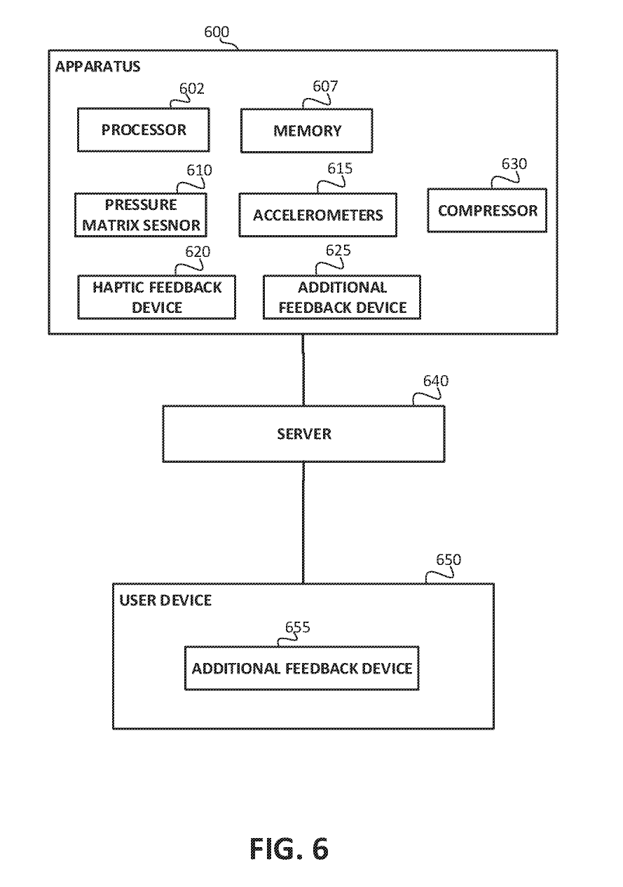

Referring now to FIG. 6 showing a block diagram of a system, in accordance with some exemplary embodiments of the disclosed subject matter.

Apparatus 600, such as Chair 100a or Chair-Mountable Pad 100b, may comprise a Processor 602. Processor 602 may be a Central Processing Unit (CPU), a microprocessor, an electronic circuit, an Integrated Circuit (IC) or the like. Processor 602 may be utilized to perform computations required by Apparatus 600 or any of it subcomponents.

In some exemplary embodiments, Apparatus 600 may comprise a Memory 607. Memory 607 may be a hard disk drive, a Flash disk, a Random Access Memory (RAM), a memory chip, or the like. In some exemplary embodiments, Memory 607 may retain program code operative to cause Processor 602 to perform acts associated with any of the subcomponents of Apparatus 600. Memory 607 may retain readings obtained from sensors, classifiers used for posture estimation and feedback selection, feedback history, or the like. Memory 607 may retain rules, selection rules, or the like, used by Apparatus 600.

Apparatus 600 may comprise Pressure Matrix Sensor 610 capable of sensing pressure in a matrix of N×M locations. In some exemplary embodiments, a first Pressure Matrix Sensor 610 may be located on the backrest and a second Pressure Matrix Sensor 610 may be located on the seat.

Apparatus 600 may comprise Accelerometers 615. In some exemplary embodiments, at least two Accelerometers 615 may be comprised by Apparatus 600 and located at opposite locations to one another in the seat, in the backrest, or the like.

Apparatus 600 may comprise Haptic Feedback Device 620. Additionally or alternatively, one or more Additional Feedback Devices 625 may be comprised by Apparatus 600. Processor 602 may be configured to process sensor readings and select a feedback device to provide feedback to the person sitting on the chair. The feedback may be provided through either one of the feedback devices 620, 625 or through additional feedback devices that are reachable over a network, such as 645.

In some exemplary embodiments, connection to the additional feedback device may be direct, such as by a direct connection to User Device 640, or indirectly, such as via Server 630.

In some exemplary embodiments, Server 630 may comprise a data repository to retain cumulative sensor reading history, feedback history, or the like. Apparatus 600 may upload data to Server 630, such as periodically, when connected thereto, or the like. Apparatus 600 may download updated rules from Server 630. Additionally or alternatively, Apparatus 600 may download updated classifiers (not shown), after Server 630 re-trains such classifiers based on monitored data.

In some exemplary embodiments, a user's mobile device, such as User Device 640, may be used to provide user feedback, such as using an instant notification, an email message sent to the user's email, a text message sent to the user's phone number, via an application program installed on the device, or the like.

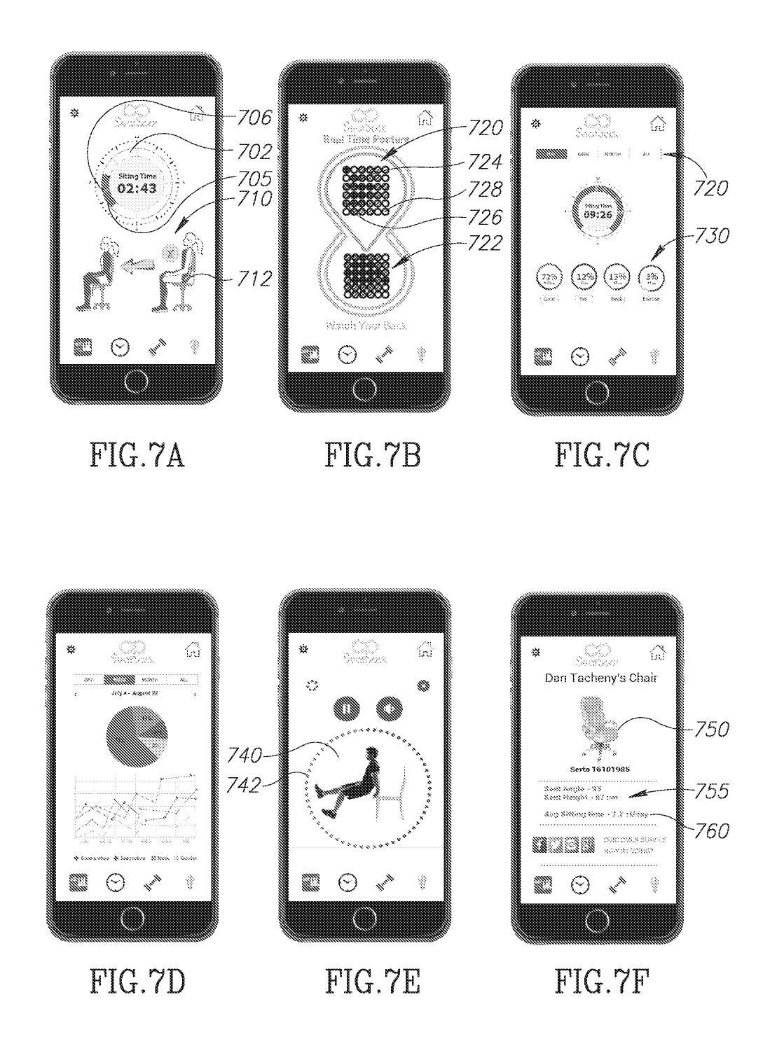

FIG. 7A shows an illustration of a user interface of an application program, in accordance with some exemplary embodiments of the disclosed subject matter. Current sitting time is displayed (702) together with a visual indication of aggregated posture information over time (706, 708). For example, indication of aggregative time in the last time window of an hour is shown for good posture (708) and rest time (706). Real-time message (710) is displayed if current estimated posture is incorrect. Real-time message (710) may indicate area of the body where the posture is incorrect (712).

Another user interface illustration is shown in FIG. 7B, which shows real time pressure map. Pressure maps corresponding to pressure sensor matrices are shown. Pressure Map 720 shows pressure sensed on backrest. Pressure Map 722 shows pressure sensed on seat. The pressure sensed by each sensor is visually displayed, such as indicating no pressure (728), and different degrees of pressure (724, 726).

Another user interface illustration is shown in FIGS. 7C and 7D, which show user analytics. In FIG. 7C, aggregated information is shown relating sitting time and times in which the different postures were detected (730). A distribution of the different postures over time is visually show. In some cases, the postures may be abstracted into groups of Good, Bad, Break and Exercise. Additionally or alternatively, the bad postures may be divided into different forms of bad postures. Time window selector 732 may control a time window for which the data is shown. FIG. 7D exemplifies an alternative display for a time granularity of days in a time window of a week.

Another user interface illustration is shown in FIG. 7E, exemplifies exercise information screen. Animation 740 may be displayed showing how the person should exercise. A Visual Timer 742 may be displayed and used to time the exercise time of the person and indicate when the person may finish exercising.

Another user interface illustration is shown in FIG. 7F. Information about the specific chair model may be shown (750). The chair model may be a-priori known if the apparatus is embedded within the chair and is permanently attached thereto. Additionally or alternatively, the user may manual indicate the model. Suggested chair setting may be displayed (755). The suggested settings may be determined based on the physical measurements of the person, based on expert knowledge relating to the chair, combination thereof, or the like. In some exemplary embodiments, the suggested setting may be displayed to the user. Additionally or alternatively, the suggested setting may be applied automatically. In some exemplary embodiments, statistical information about the usage of the chair, such as average sitting time (760) may be displayed.

### An Embodiment

One embodiment of the disclosed subject matter may be a device as described hereinbelow. The device checks every about 2 sec if the user sitting. Such a check may not be performed if the device is in sleep mode. If the user is detected as sitting, sitting time is incremented. Otherwise, the sitting time may be reset to zero. Before resetting the data may be backed up and saved on a flash drive.

The device may compare current detected posture to predetermined postures database. The current posture may be noted and a counter for the posture is incremented to track statistics of postures.

The device may check if the user sits for a period of time than more 50 minutes. If so, and if a number of alerts issued so far is below three, and the last alerts was issued at least 30 seconds before, the device provides a bad posture feedback to the user. Otherwise, the user is determined to be non-responsive, an alerts may be turned off for 60 minutes.

In some exemplary embodiments, the device may check if the posture is a good posture and last alert was issued before the last time in which the posture was good or last time where the user was standing. If so, alerting is turned off. Good posture sitting time is counted. If a threshold of 20 seconds is reached, a sit good label is determined.

Otherwise, if the posture is not a good posture, and if a number of alerts issued so far is below three, and the last alerts was issued at least 30 seconds before, the device provides a bad posture feedback to the user. Otherwise, the user is determined to be non-responsive, an alerts may be turned off for 60 minutes.

If the device determines that the user stands for more than 1 minutes, the device saves all the parameters from the user sitting session on the Flash memory. The device may track the time the user sits in his current posture.

If the user stand for more than 20 minutes, the device may go to sleep mode and check whether the user started sitting on it every 30 seconds.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

1-24. (canceled)

25. A system comprising:
at least two accelerometer sensors, a first one of the at least two accelerometer sensors positioned on a first surface of a chair, and a second one of the at least two accelerometers positioned on a second surface of the chair, the first and second surfaces being at an angle with respect to each other;
a processor configured to process readings from the at least two accelerometers and
(i) to compute a first average angle between the first surface of the chair and a plane parallel to a ground, and a second average angle between the second surface of the chair and the plane parallel to the ground,
(ii) to calculate based on the first and second average angles, an angle indicative of an angle between a lumbar area and a thigh of a person sitting on the chair, and
(iii) to determine the posture of the person based on the calculation of the indication of the angle between the lumbar area and the thigh of the person; and
a feedback device configured to receive instructions from the processor and provide a feedback signal to the person based on the determined posture.

26. The system according to claim 25, wherein the first surface of the chair comprises a backrest of the chair and the second surface of the chair comprises a seat of the chair.

27. The system according to claim 26, wherein:
the first one of the at least two accelerometers is positioned at a top portion of the backrest and is configured to provide readings relating to a spatial orientation of the backrest with respect to the vector of gravity; and
the second one of the at least two accelerometers is positioned at an external border of the seat and is configured to provide readings relating to a spatial orientation of the seat with respect to the vector of gravity.

28. The system according to claim 26, further comprising a third accelerometer and wherein the processor is configured to calculate the angle indicative of the angle between the lumbar area and the thigh of the person, based on readings from the first, second and third accelerometers.

29. The system according to claim 28,
wherein the third accelerometer is positioned in proximity to an intersection between the backrest and the seat of the chair and is configured to provide readings relating to a spatial orientation of the seat with respect to the vector of gravity, and
wherein the processor is configured to compute an average angle between the backrest and the seat of the chair based on the readings from the third accelerometer with respect to the readings from the first and second accelerometers.

30. The system according to claim 25, further comprising a set of pressure sensors, and wherein the processor is configured to determine the posture of the person based on the readings from the set of pressure sensors.

31. The system according to claim 30, wherein the first surface of the chair comprises a backrest of the chair and the second surface of the chair comprises a seat of the chair, and wherein the set of pressure sensors are arranged in a first matrix of pressure sensors located on the seat of the chair, and a second matrix of pressure sensors located on the backrest of the chair.

32. The system according to claim 25, further comprising a set of pressure sensors, and wherein the processor is configured to determine the posture of the person based on the readings from the pressure sensor in combination with the readings from the at least two accelerometers.

33. The system according to claim 25, wherein the feedback device comprises a haptic feedback device located in the chair and configured to provide haptic feedback to the person based on the estimated posture.

34. The system according to claim 33, wherein the feedback device is configured to provide different haptic feedbacks using the haptic feedback device or using an additional feedback device located externally to the chair.

35. The system according to claim 25, wherein the feedback device is configured to provide the person with a first feedback based on a first estimated posture and a second feedback based on a second estimated posture; wherein the first feedback is a negative feedback inducing posture modification; and wherein the second feedback is a positive feedback that is provided in response to posture modification in response to the first feedback.

36. A method comprising:
positioning at least two accelerometer sensors, a first one of the at least two accelerometer sensors on a first surface of a chair, and a second one of the at least two accelerometers on a second surface of the chair, the first and second surfaces being at an angle with respect to each other;
using a processor, processing readings from the at least two accelerometers and
(i) computing a first average angle between the first surface of the chair and a plane parallel to a ground, and a second average angle between the second surface of the chair and the plane parallel to the ground,
(ii) calculating based on the first and second average angles, an angle indicative of an angle between a lumbar area and a thigh of a person sitting on the chair, and
(iii) determining the posture of the person based on the calculation of the indication of the angle between the lumbar area and the thigh of the person; and
using a feedback device, providing a feedback signal to the person based on the determined posture.

37. The method according to claim 36, wherein the first surface of the chair includes a backrest of the chair and the second surface of the chair includes a seat of the chair, and wherein positioning comprises positioning the first one of the at least two accelerometer sensors at a top portion of the backrest and the second one of the at least two accelerometers at an external border of the seat.

38. The method according to claim 36, further comprising using a third accelerometer and wherein using the processor comprises using the processor to calculate the angle indicative of the angle between a lumbar area and a thigh of the person, based on readings from the first, second and third accelerometers.

39. The method according to claim 38, wherein the first surface of the chair includes a backrest of the chair and the second surface of the chair includes a seat of the chair, and wherein the method further comprises positioning the third accelerometer in proximity to an intersection between the backrest and the seat of the chair.

40. The method according to claim 36, further comprising positioning a set of pressure sensors on the chair, and wherein determining the posture of the person further comprises determining the posture based on readings from the pressure sensors and from the at least two accelerometers.

41. The method according to claim 40, wherein the first surface of the chair includes a backrest of the chair and the second surface of the chair includes a seat of the chair and positioning the set of pressure sensors comprises arranging the pressure sensors in a first matrix of pressure sensors located on the seat of the chair, and a second matrix of pressure sensors located on the backrest of the chair.

42. The method according to claim 41, wherein providing a feedback signal to the person, comprises providing the subject with a pressure map generated based on the readings of the first and second matrices of pressure sensors and showing the pressure sensed on the backrest and on the seat.

43. The method according to claim 36, wherein providing the feedback comprises providing the person a first feedback based on a first estimated posture and a second feedback based on a second estimated posture; wherein the first feedback is a negative feedback inducing posture modification; and wherein the second feedback is a positive feedback that is provided in response to posture modification in response to the first feedback.
